(19) United States
(12) Patent Application Publication
(10) Pub. No.: US 2019/0008458 A1
(43) Pub. Date: Jan. 10, 2019

(54) DEVICE AND METHOD FOR EXTRACTING HEART RATE INFORMATION

(71) Applicant: KONINKLIJKE PHILIPS N.V., EINDHOVEN (NL)

(72) Inventors: Cornelus Hendricus Bertus Arnoldus van Dinther, Mierlo (NL); Cornelis Harm Taal, Utrecht (NL)

(21) Appl. No.: 16/074,885

(22) PCT Filed: Feb. 14, 2017
(86) PCT No.: PCT/EP2017/053262

§ 371 Date: Aug. 2, 2018

(30) Foreign Application Priority Data
Feb. 15, 2016 (EP) 1655698.0

(51) Int. Cl. A61B 5/00; A61B 5/024

(52) U.S. Cl. CPC A61B 5/721; A61B 2562/0219; A61B 5/7253; A61B 5/02416

(57) ABSTRACT

A data processing device (100, 200) determines heart rate information regarding a subject of interest (605) from time-dependent sensor data (110) that includes physiological sensor data (114) comprising a heart beat component and at least one motion artifact component, and that further includes motion sensor data (116). A reconstruction unit receives artifact-removed physiological sensor data (130) and decomposes it into a plurality of artifact-removed physiological sensor data components (145) with associated component amplitudes, and provides reconstructed heart beat component data (150) as a combination of a component subset of at least two of the artifact-removed physiological sensor data components (145) of highest component amplitudes. A beat analysis unit receives the reconstructed heart beat component data (150) and determines an interbeat interval from the heart beat component data (150) as the heart rate information, and provides a heart rate information signal (170) based on the determined heart rate information.

### FIELD OF THE INVENTION

The invention relates to a data processing device for determining heart rate information regarding a subject of interest from time-dependent sensor data that includes physiological sensor data comprising a heart beat component and at least one motion artifact component, and that further includes motion sensor data, which is indicative of a velocity or an acceleration of a sensed region of the subject of interest. It further relates to an apparatus for determining heart rate information regarding a subject of interest, to a data processing method and to a computer program.

### BACKGROUND OF THE INVENTION

Information about cardiovascular status, such as a heart rate of a subject of interest can be unobtrusively acquired by photoplethysmography (PPG) using sensors such as contact sensors or remote sensors such as a camera. A PPG technique using a remote sensor is also referred to as remote PPG.

Whether using contact sensors or remote sensors, the PPG technique is susceptible to motion-induced signal distortions, which are superimposed to the desired vital signal. Motion-induced signal distortions for instance arise from motion of the subject of interest. Motion artifact reduction in PPG data representing the detected PPG signals is a challenging task since the contribution of the motion components often exceeds the contribution of the desired vital signal by an order of magnitude. The artifacts lead to erroneous interpretation and degrade the accuracy and reliability of estimation of cardiovascular parameters.

In a number of studies the associated PPG setups were usually operated under conditions that required the subjects to be motionless. This drawback limits the capabilities of the technique in real application environments, e.g. hospital, home care and sports.

US 2014/0213858 A1 discloses a portable device for determining a heart rate of a person, comprising a heart rate measurement unit, a motion measurement unit for measuring the motion of a body part, and a processing unit. The processing unit is adapted to measure a signal quality of the heart rate signal and accordingly to switch between two calculation modes: if the signal quality is above a predefined threshold, the heart rate is calculated based on the heart rate signal. If the signal quality is so poor that a reliable calculation of the heart rate is technically not possible anymore based on the heart rate signal, the processing unit switches to its second calculation mode, in which the heart rate is estimated based on the motion signal by estimating a heart rate constant, which depends on the frequency of the motion signal, and defining an exponential development of the heart rate, starting at the last reliably measured heart rate and finishing at the estimated heart rate constant.

US 2012/0229201 A1 describes a filter device which includes a filter that separates a steady component and a non-steady component included in an input signal, a synthesis unit that synthesizes the separated steady component and the separated non-steady component according to a given ratio, and an evaluation unit that evaluates the magnitude of the amount of the non-steady component in the input signal, wherein the synthesis unit sets the given ratio to a first ratio in an instance in which the evaluation unit determines the amount of the non-steady component to be equal to or less than a predetermined reference, and sets the given ratio to a second ratio, in which the proportion of the non-steady component is less than that of the first ratio, in an instance in which the evaluation unit determines the amount of the nonsteady component to be greater than the predetermined reference.

### SUMMARY OF THE INVENTION

It would be desirable to provide reliable and more accurate heart rate information from physiological sensor data even s in presence of strong motion-induced signal distortions.

According to a first aspect of the invention, a data processing device for determining heart rate information regarding a subject of interest from time-dependent sensor data is provided. The time-dependent sensor data includes physiological sensor data comprising a heart beat component and at least one motion artifact component, and further includes motion sensor data, which is indicative of a velocity or an acceleration of a sensed region of the subject of interest. The data processing device comprises:
a motion-artifact removal unit, configured to receive the sensor data, to apply a motion-artifact-removal algorithm to the physiological sensor data using the motion sensor data and to provide artifact-removed physiological sensor data;
a reconstruction unit, configured to receive the artifact-removed physiological sensor data and to decompose the artifact-removed physiological sensor data into a plurality of artifact-removed physiological sensor data components with associated component amplitudes, and to provide reconstructed heart beat component data as a combination of a component subset of at least two of the artifact-removed physiological sensor data components of highest component amplitudes; and
a beat analysis unit, configured to receive the reconstructed heart beat component data, to determine an interbeat interval from the heart beat component data as the heart rate information, and to provide a heart rate information signal based on the determined heart rate information.

The data processing device of the first aspect of the present invention is based on the recognition that a data processing stage for motion-artifact removal alone may not always allow a reliable determination of heart rate information from physiological sensor data, in particular in the presence of strong distortions due to motion artifacts. The inventors have found that artifact-removed physiological sensor data may have remaining irregularities that would not be present in sensor data obtained from a resting subject of interest. Techniques for deriving heart rate information that rely strictly on a shape of the pulse as a function of time may therefore not able to extract a heart beat component from the physiological sensor data reliably. In this sense, it becomes clear that the term “motion-artifact removal” shall not be understood in the context of the present specification to imply a complete and reliable removal of motion artifacts from the physiological sensor data. The term is used here to denote a technical approach of data processing for reducing motion-induced distortions in the physiological sensor data. The data processing device of the first aspect of the present invention serves to advance the technology by further reducing limitations of existing approaches of artifact removal, which under some circumstances leave irregularities in the nominally artifact-removed physiological sensor data.

To cope with such irregularities, the present invention suggests providing a second processing stage for quasi reconstructing the heart beat component from the artifact-removed physiological sensor data. In this context, the invention is furthermore based on the recognition that a reliable reconstruction of the heartbeat component can be achieved efficiently even under strong original distortion by motion artifacts by a decomposition of the artifact-removed physiological sensor data into a set of component with associated respective component amplitudes, followed by a subsequent reconstruction of the heart beat component using only a component subset formed by those artifact-removed physiologic sensor data components with highest component amplitudes. Since the reconstructed heart beat component has no phase distortion, an accurate beat detection is ensured. This way, a periodic interbeat interval can be reliably and accurately determined in order to provide the heart rate information signal.

The data processing device according to the first aspect can thus advantageously minimize errors of the determined interbeat interval values and thus provide precise heart rate information even at a high motion level when strong motion induced distortions are present in the physiological sensor data.

A further advantage of the data processing device is that it does not require the use of previously recorded sensor data for motion-artifact removal and reconstruction of the heart beat component, such as other techniques that apply recursive methods.

This allows embodiments making use of a frame-by-frame based determination of the heart rate information, particularly enabling real-time processing to provide a desirable immediate output of heart rate information to a user.

The data processing device is particularly suited to be operated in combination with PPG apparatus for use in a hospital or in sports. This way, the data processing device of the first aspect of the present invention forms a key to an increased use of PPG apparatus in medical environments as well as every-day life.

In the following, embodiments of the data processing device according to the first aspect of the invention will be described.

In a preferred embodiment, the data processing device is further configured to receive from an external motion sensor a motion level indicative of a current amount of velocity or acceleration of the sensed region of the subject of interest. The sensed region is typically a small body region of the subject of interest and comprises a section of skin and underlying tissue including pulsating blood vessels in a suitable body part, the body part being for instance a finger, arm, or leg of a subject of interest.

In this embodiment the beat analysis unit is further arranged and configured to determine from the motion sensor data or to receive a motion level indicator indicative of whether a motion level derived from a current amount of velocity or acceleration of the sensed region of the subject of interest exceeds a predetermined motion level threshold; and the beat analysis unit is further arranged and configured, only as long as according to the motion level indicator the motion level does not exceed a predetermined motion level threshold, to receive the physiological sensor data and to determine the interbeat interval from only the physiological sensor data.

In this embodiment, the beat analysis unit has additional functionality to extract the heart rate information directly from the artifact-removed sensor data under suitable conditions, so that a processing time and capacity required by the motion-artifact removal unit and the reconstruction unit can be avoided.

In one variant of this embodiment, the data processing unit is configured to receive the motion level and to switch the motion-artifact removal unit and the reconstruction unit to an inactive state, when the motion level is below the predetermined threshold, and to switch these units into an active state, when the motion level is above the predetermined threshold.

In an alternative of this embodiment, the data processing unit is configured to keep the motion-artifact removal unit in operation even as long as the motion level is below the threshold motion level, and to switch only the reconstruction unit in to the inactive state, when the motion level falls below the threshold motion level.

The motion level can be determined for instance in dependence on signal amplitudes in a predetermined time interval of the motion sensor data or in dependence on amplitudes of certain frequency components in a frequency spectrum of motion derived from the motion sensor data.

In a further preferred embodiment, the data processing device is configured to receive the physiological sensor data in form of PPG data, which is indicative of an amount of electromagnetic radiation reflected from or transmitted through the sensed region of the subject of interest as a function of time in at least one first spectral channel that is sensitive to blood volume variations in the sensed region. Preferably, the at least one first spectral channel includes electromagnetic radiation having a wavelength between 500 nm and 600 nm. In some variants of this embodiment, the at least one spectral channel covers wavelengths in a spectral interval between 530 nm and 570 nm, or between 540 nm and 560 nm. Preferred embodiments include the wavelength of 550 nm, which provides a particularly high sensitivity to blood volume variations.

In an embodiment according to the first aspect of the invention the data processing device is configured to receive the time dependent motion sensor data at least partly in form of optical motion sensor data, which is indicative of an amount of electromagnetic radiation reflected from or transmitted through the sensed region of the subject of interest as a function of time in at least one further spectral channel that is not sensitive to blood volume variations in the sensed region. The at least one further spectral channel may for example contain a wavelength interval around the wavelength of 650 nm, e.g., 610 nm-700 nm, which has a relatively lower pulsatility due to blood volume variations in the skin. In other variants, the motion sensor data contains optical motion sensor data in more than one further spectral channel, for instance in two or three spectral channels. In addition to the mentioned suitable spectral channel covering a wavelength interval around the wavelength of 650 nm, a further spectral channel may be comprised in the optical motion sensor data that covers a wavelength interval around a center wavelength shorter than 550 nm, such as for instance a center wavelength of 450 nm.

In a further embodiment, the data processing device is configured to receive the time dependent motion sensor data at least partly in form of acceleration data provided by an accelerometer. Accelerometers are well known and widely used in mobile electronic devices, such as mobile phones, and therefore are not further discussed in detail herein. In a further variant, the motion sensor data comprises optical motion sensor data as well as accelerometer data. The accelerometer data may only be used to determine a motion level value, while the optical motion sensor data is decomposed within the motion artifacts removal unit, or vice versa. Using optical motion sensor data as well as accelerometer data as motion sensor data can allow a further increasing the reliability of the heart rate information determined by data processing unit.

In preferred embodiments, the data processing device is further configured to structure the time-dependent sensor data into frames containing sensor data pertaining to predetermined time spans. The frames may in different ones of these embodiments either overlap in time or strictly partition the data in disjoint time intervals. In embodiments that are suitable in particular for real-time processing, a frame may represent a concatenation of the incoming sensor data and a number of seconds of sensor data from the recent past A frame of sensor data, such as PPG data and/or accelerometer data, is thus to be understood as a data structure containing the time-dependent sensor data pertaining to a predetermined time interval of a time base. For instance, a frame may cover a number of seconds, in particular less than 10 seconds, preferably less than 5 seconds, and for instance at least 2 seconds. These given values are exemplary and may be varied in dependence on a sampling rate of the sensor data. In a variant, the frame covers at least a duration of one complete period of a heart beat cycle.

In one implementation of this embodiment, the motion-artifact removal unit is further configured to decompose the motion sensor data and to combine the physiological sensor data and the sets of motion reference data on a frame-by-frame basis. Furthermore, it is preferred that also the reconstruction unit is configured to decompose the artifact-removed physiological sensor data and to combine the component subset of the at least two of the artifact-removed physiological sensor data components on a frame-by-frame basis. In this embodiment in particular, no reference to previously recorded sensor data, i.e., sensor data recorded prior to the currently processed frame, is made for decomposing a and reconstructing a current frame.

Another embodiment has the reconstruction unit in a configuration that is operative to adapt a total number of the artifact-removed physiological sensor data components forming the component subset. Adaptation is in one implementation performed in the course of presetting operational parameters, and made for instance in dependence on a type of motion sensor data used by the data processing device, e.g. optical motion sensor data or accelerator data, or in dependence on a selected sensed region of the subject of interest. In another implementation that may be used alone or in combination with the first mentioned implementation, the adaptation is performed dynamically in the course of operation of the data processing device. This latter implementation allows maintaining a desired high level of reliability of heart beat analysis under different conditions regarding the determined component amplitudes in the component spectrum.

Regarding criteria for use to control the adaptation of the components used for reconstruction entering beat detection (herein also referred to as predetermined adaptation control criteria), the motion level, a shape of the component spectrum, e.g., having more than two similar component amplitudes, or, in a loop, an embedded dimension K or a window length N could be used dynamically. Thus, one embodiment has the reconstruction unit operative to adapt the total number of the artifact-removed physiological sensor data components forming the component subset by changing this total number in dependence on a current motion level, or a current shape of the component spectrum.

Different technologies are available and per se known for implementing the motion-artifact removal unit. In one embodiment, the motion-artifact removal unit is configured to decompose the motion sensor data into at least two components of decomposed motion sensor data, to determine, based on the at least two components of decomposed motion sensor data, at least two different sets of motion reference data, and to combine the physiological sensor data and the sets of motion reference data so as to provide the artifact-removed physiological sensor data. Preferably, the motion-artifact removal unit is configured to decompose the motion sensor data into the at least two components of decomposed motion sensor data using a singular spectrum analysis algorithm. Singular spectrum analysis is a technique which is known per se in the art.

In a further embodiment, the motion sensor data is decomposed with respect to its frequency components or phase components, so as to provide the at least two components of decomposed motion sensor data in the form of at least two respective frequency components or at least two respective phase components of the motion sensor data. In a variant of this embodiment, the motion-artifact removal unit is further configured such that the respective frequency components or phase components of the motion sensor data might further be shifted by using a Hilbert transform algorithm and providing Hilbert transformed data as decomposed motion sensor data.

In a preferred embodiment, also the reconstruction unit is configured to decompose the artifact-removed physiological sensor data into the plurality of artifact-removed physiological sensor data components using a singular spectrum analysis algorithm. In one implementation of this embodiment, the reconstruction unit is configured to determine eigenvalues and eigenvectors of a covariance matrix pertaining to a trajectory matrix, which has column vectors of artifact-removed physiological sensor data samples of a time frame of predetermined length, and to project the trajectory matrix onto the eigenvectors so as to determine the artifact-removed physiological sensor data components and their associated component amplitudes. In this embodiment the eigenvalues determined in the course of the singular spectrum analysis algorithm thus determine the component amplitudes of the artifact-removed physiological sensor data components.

The data processing device can be provided in small physical dimensions. It is therefore suited for use in an apparatus for determining a heart rate of a subject of interest that is a wearable device, such as chest belt PPG device, wrist watch PPG device or a finger PPG device or a toe PPG device.

According to a second aspect of the invention, thus, an apparatus for determining a heart rate of a subject of interest is provided. The apparatus comprises
an emitter unit, which comprises at least one emitter, which is configured to emit electromagnetic radiation at a sensed region of the subject of interest in at least one spectral channel that allows ascertaining physiological sensor data comprising a heart beat component,
a sensor unit, which is configured to ascertain and provide at its output physiological sensor data that is indicative of an amount of electromagnetic radiation reflected from or transmitted through a sensed region of the subject of interest as a function of time in at least one spectral channel and that includes the heart beat component and at least one motion artifact component, and to ascertain motion sensor data that is indicative of a velocity or an acceleration of the sensed region as a function of time; and
a data processing device according to the first aspect of the invention or one of its embodiments.

The apparatus of the second aspect of the invention shares the advantages of the data processing device of the first aspect of the invention.

Embodiments of the apparatus that are particularly suited for use in every-day life and sports make use of PPG data taken in different spectral ranges. Such devices can be particularly compact. In such embodiments, the emitter unit is additionally configured to emit electromagnetic radiation in at least one further spectral channel that is less sensitive to blood volume variations in the sensed region than the first spectral channel. The sensor unit is additionally configured to ascertain PPG data indicative of an amount of electromagnetic radiation reflected from or transmitted through the sensed region in the at least one further spectral channel.

Alternatively or additionally to making use of the additional spectral channel of PPG data, an accelerometer can be provided as a part of the sensor unit. In an embodiment, the accelerometer is arranged and configured to provide at least a part of the motion sensor data in form of accelerometer data. Accelerometers based on semiconductor technology are widely used in handheld device today and can be provided in very compact size.

According to a third aspect of the invention, a data processing method for extracting heart rate information of a subject of interest from time-dependent sensor data is provided. The data processing method comprising:
receiving time-dependent sensor data that includes physiological sensor data comprising a heart beat component and at least one motion artifact component, and that further includes motion sensor data, which is indicative of a velocity or an acceleration of a sensed region of the subject of interest;
applying a motion-artifact-removal algorithm to the physiological sensor data using the motion sensor data and providing artifact-removed physiological sensor data;
receiving the artifact-removed physiological sensor data, decomposing the artifact-removed physiological sensor data into a plurality of artifact-removed physiological sensor data components with associated component amplitudes, combining a component subset comprising at least two of the artifact-removed physiological sensor data components with highest component amplitudes, and providing the combined component subset as reconstructed heart beat component data; and
receiving the reconstructed heart beat component data, determining an interbeat interval from the heart beat component data as the heart rate information, and providing a heart rate information signal based on the determined heart rate information.

The data processing method of the third aspect of the invention shares the advantages of the data processing device of the first aspect of the invention.

In the following, some of the embodiments of the method will be explained.

Applying a motion-artifact-removal algorithm preferably comprises decomposing the motion sensor data into at least two components of decomposed motion sensor data, determining at least two different sets of motion reference data, based on the at least two components of decomposed motion sensor data, combining the physiological sensor data and the sets of motion reference data, and providing artifact-removed physiological sensor data.

In an embodiment, the method further comprises:
ascertaining of a motion level indicative of a current amount of velocity or acceleration of the sensed region of the subject of interest; and,
only as long as the motion level value does not exceed a predetermined motion level threshold: determining the interbeat interval from only the sensor data or the physiological sensor data.

According to a fourth aspect of the present invention, a computer program comprises program code means for causing a computer to perform the method according to the third aspect of the invention or one of its embodiments when said computer program is executed on the computer.

The computer which executes the computer program may for instance be a data processing device in the form of a microcontroller or microprocessor. The computer can be a computer watch device. In another embodiment, the computer forms an integrated part of a hospital computer system. In yet another embodiment, the computer is integrated into a medical device and the computer program comprises program code means for determining further vital sign information, such as respiratory rate, blood pressure, blood volume fraction and oxygen saturation from the sensor data received by the device.

It shall be understood that the data processing device of the first aspect of the invention, also defined in claim 1, the apparatus of the second aspect of the invention, also defined in claim 12, the data processing method of the third aspect, also defined in claim 14, and the computer program according to the fourth aspect, also defined in claim 15, have similar or identical embodiments.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows a block diagram of a first embodiment of the data processing device according to a first aspect of the invention;

FIG. 2 shows a block diagram of a second embodiment of the data processing device according to the first aspect of the invention;

FIG. 3 shows a block diagram of an embodiment of a motion-artifact removal unit for use in the first or second embodiment of the data processing device;

FIG. 4 shows a block diagram of an embodiment of a reconstruction unit for use in the first or second embodiment of the data processing device;

FIGS. 5a-d show sensor data (FIG. 5a), artifact-removed physiological sensor data (FIG. 5b), reconstructed heart beat component (FIG. 5c) and an electrocardiogram (FIG. 5d) over a time period of 5 sec received at respective units of the data processing device according to the first aspect of the invention;

FIG. 6 shows an embodiment of a PPG apparatus according to a second aspect of the invention;

FIG. 7 shows a schematic illustration of a first embodiment of a data processing method according to a third aspect of the invention;

FIG. 8 shows a schematic illustration of a second embodiment of the data processing method according to the third aspect of the invention.

### DETAILED DESCRIPTION OF EMBODIMENTS

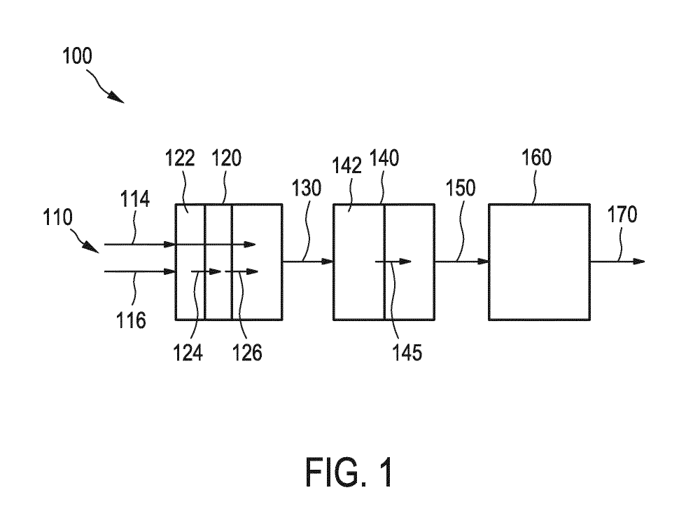

FIG. 1 shows a block diagram of a first embodiment of the data processing device 100 according to a first aspect of the invention.

The data processing device 100 is configured to extract a heart rate information regarding a subject of interest from time-dependent sensor data 110 that includes physiological sensor data 114 comprising a heart beat component and at least one motion artifact component, and that further includes motion sensor data 116, which is indicative of a velocity or an acceleration of a sensed region of the subject of interest.

The data processing device 100 comprises a motion-artifact removal unit 120 providing artifact-removed physiological sensor data 130, a reconstruction unit 140 providing reconstructed heart beat component data 150 and a beat analysis unit 160 provide a heart rate information signal based on the determined heart rate information. The mentioned units will be described in more detail in the following.

The motion-artifact removal unit 120 is configured to receive the sensor data 110, to decompose the motion sensor data 116 into at least two components of decomposed motion sensor data 124, to determine, based on the at least two components of decomposed motion sensor data 124, at least two different sets of motion reference data 126 and to combine the physiological sensor data 114 and the sets of motion reference data 126 so as to provide artifact-removed physiological sensor data 130. The decomposing of the motion sensor data 116 is provided by a first singular spectrum analysis unit 122 that forms a part of the motion-artifact removal unit 120 and uses a singular spectrum analysis algorithm.

The reconstruction unit 140 is configured to receive the artifact-removed physiological sensor data 130 and to decompose the artifact-removed physiological sensor data 130 into a plurality of artifact-removed physiological sensor data components 145 with associated component amplitudes, and to provide reconstructed heart beat component data 150 as a combination of a subset of at least two of the artifact-removed physiological sensor data components 145 of highest component amplitudes. The decomposition of the artifact-removed physiological sensor data 130 is provided by a second singular spectrum analysis unit 142 that forms a part of the reconstruction unit 140 and uses a singular spectrum analysis algorithm.

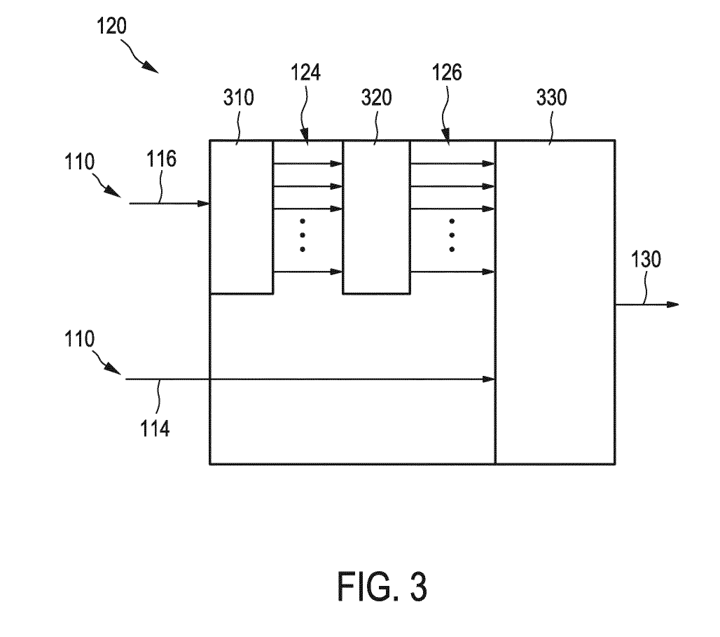

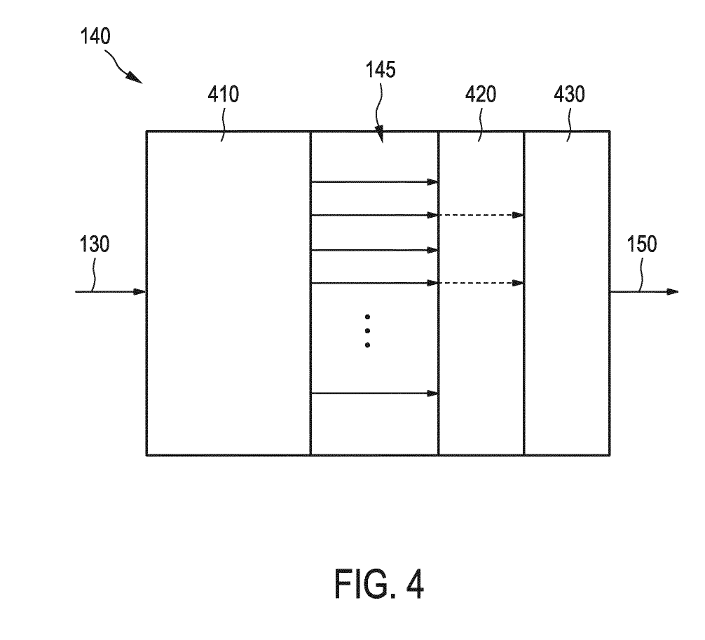

The functionality of the first and second singular spectrum analysis unit 122, 142 is described in more detail further below in the context of FIG. 3 and FIG. 4.

The beat analysis unit 160 is configured to receive the reconstructed heart beat component data 150, to determine an interbeat interval from the heart beat component data 150 as the heart rate information, and to provide a heart rate information signal 170 based on the determined heart rate information.

The motion artifact unit 120, the reconstruction unit 140 and the beat analysis unit 160 are configured to process the respectively received data as frames pertaining to predetermined time spans. In particular, the motion-artifact removal unit 120 is configured to decompose the motion sensor data 116 and to combine the physiological sensor data 114 and the sets of motion reference data 126 on a frame-by-frame basis. Furthermore, the reconstruction unit 140 is configured to decompose the artifact-removed physiological sensor data 130 and to combine a subset comprising at least two of the artifact-removed physiological sensor data components 145 on a frame-by-frame basis.

The data processing device 100 of the depicted first embodiment is configured to receive the physiological sensor data 114 in form of PPG data. The PPG data is indicative of an amount of electromagnetic radiation reflected from or transmitted through the sensed region of the subject of interest in at least one first spectral channel that is sensitive to blood volume changes in the sensed region. As such the PPG data contains information on the blood volume in the sensed region for a given moment in time and, taken over time and provided with associated time information, provides information on blood volume changes based on the heart beat. A suitable spectral region for the first spectral channel is for instance 530 nm to 570 nm. This spectral channel includes the wavelength region between 540 nm and 560 nm, which provides a particularly high sensitivity to blood volume variations. However, a spectral channel narrower than this can also be used. The better the first spectral channel overlaps with the known characteristic optical absorption and reflection features of blood in the spectral region between 520 and 600 nm, the better is a signal-to-noise ratio of the PPG data forming the physiological sensor data 114.

The motion sensor data 116 may be provided to the data processing device 100 in form of acceleration data provided by an accelerometer (not shown in FIG. 1) or in the form of optical motion data, which is indicative of an amount of electromagnetic radiation reflected from or transmitted through the sensed region of the subject of interest as a function of time in at least one further spectral channel that is not sensitive to blood volume variations in the sensed region. A suitable example for the further spectral channel is provided by wavelength range substantially around 650 nm, e.g., 610 nm-700 nm. This spectral channel provides a low pulsatility due to blood volume variations in the tissue. Another less sensitive and thus suitable spectral channel covers wavelengths substantially around 450 nm.

In an embodiment not shown, the sensor data is received as a single data stream by the data processing device and the data processing device is further configured to determine and separate stream into two sub-streams formed by the physiological sensor data 114 and the motion sensor data 116. This can be achieved using measures such as providing source identifiers associated with the sensor data, a first source identifier for each frame of motion sensor data, and a second source identifier for each frame of physiological sensor data.

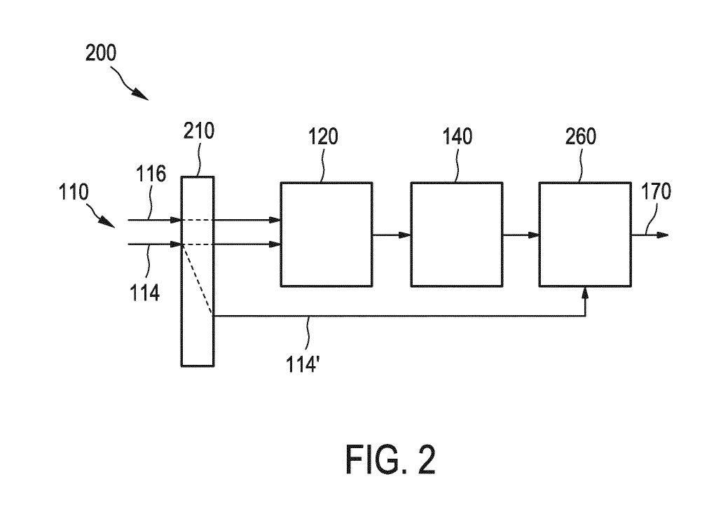

FIG. 2 shows a block diagram of a second embodiment of the data processing device 200 according to the first aspect of the invention.

The following description focuses on differences in comparison with the data processing device 100 of FIG. 1. The data processing device 200 further comprises a processing control unit 210, which is configured to determine a motion level indicative of a current amount of velocity or acceleration of the sensed region of the subject of interest from the motion sensor data 116. The processing control unit is further configured to determine a motion level indicator indicative of whether the motion level derived from the current amount of velocity or acceleration of the sensed region exceeds a predetermined motion level threshold.

Furthermore, the beat analysis unit 260 differs from the beat analysis unit 160 shown in FIG. 1 in that it is further arranged and configured to receive the physiological sensor data directly, without prior processing by the motion-artifact removal unit 120 and by the reconstruction unit 140. Thus, the beat analysis unit is additionally configured to determine the interbeat interval from only the physiological sensor data 114′.

In operation, the processing control unit 210 sets the mode of operation of the data processing device. It instructs the beat analysis unit 260 operation according to a first mode that involves providing the physiological sensor data directly to the beat analysis unit, without prior processing by the motion-artifact removal unit 120 and by the reconstruction unit 140. The processing-control unit 210 monitors the motion level indicator over time and maintains this first mode of operation for only as long as the motion level value does not exceed a predetermined motion level threshold. In case the motion level is found by the processing control unit 210 to exceed the predetermined motion level threshold, the data processing device 200 is switched to a second mode of operation, which is as previously described for the first embodiment of the data processing unit 100.

Generally, the data processing performed by the beat analysis unit for determining the interbeat interval is the same in the first and second modes of operation. However, the determination of the interbeat interval is done using different data, namely the physiological sensor data in the first mode of operation, and the reconstructed heart beat component data in the second mode of operation.

The processing-control unit 210 determines the motion level ascertaining an amplitude of the motion sensor data indicative of a maximum amount of velocity or acceleration with respect to a given time frame. Alternatively, the motion level may be determined by determining an average amount of velocity or acceleration of the sensed region of the subject of interest. A measure combining both velocity and acceleration amounts may be used in another variant. Another metric for determining the motion level could be the well-known L1-norm, i.e., a sum of absolute values of acceleration in different spatial directions, for instance in the three spatial directions, e.g., over XYZ frames, if a triaxial accelerometer is used.

FIG. 3 shows a block diagram of an embodiment of a motion-artifact removal unit 120 for use in the first or second embodiment of the data processing device 100, 200.

The motion sensor data 116 is received by the artifact removal unit 120 and fed to a first subunit 310. Here, the motion sensor data 116 is decomposed into a plurality of components 124 of decomposed motion sensor data 124. The following steps are used to compute the components of decomposed motion sensor data rₖ (n) 124 from the motion sensor data 116 s(n) within a time-frame that leads to the range n ∈ {1, . . . ,N}. Based on an embedded dimension K and the definition L:=N+1−K, a L×K Hankel matrix S=[s₀,s₁, . . . ,s_{K}] is formed. Here, sₖ are column vectors with elements sₖ:=[s(k),s(k+1), . . . ,s(k+L−1]^{T}. Based on the determined Hankel matrix S, eigenvalues λ₁≥λ≥. . . ≥λ_{K}≥0 and eigenvectors vₗ, . . . ,v_{K} of the covariance matrix S^{T}S are determined. Afterwards, S is projected onto the eigenvectors A:=SV, where A is a matrix containing the principal components aₖ as columns and V a matrix with eigenvectors vₖ as columns. The components of decomposed motion sensor data rₖ(n) 124 are determined by the equation

$${r}_{k} ( n ) := \frac{1}{{M}_{n}} {\sum }_{m = {L}_{n}}^{{U}_{n}} {a}_{k} ( n - m + 1 ) {v}_{K} ( m ) ,$$

with (Mₙ, Lₙ, Uₙ)=(1/ n,1, n) for 1≥n≥M−1, (Mₙ, Lₙ, Uₙ)=(1/M,1,M) for M≥n≥K and (Mₙ, Lₙ, Uₙ)=(N−n+1) , n−N+M, M) for K+1≥n≥N.

A second removal subunit 320 determines the different sets of motion reference data 126, based on the components of decomposed motion sensor data 124. In the present embodiment, the motion reference data 126 is identical to the decomposed motion sensor data 124. In embodiments not shown, the motion reference data comprises respective subsets of the decomposed motion sensor data. In a further embodiment not shown, the motion reference data comprises time-shifted decomposed motion reference data.

A third removal subunit 330 is configured to receive the physiological sensor data 114 and the motion reference data 126. The artifact-removed physiological sensor data 130 d(k) is provided by combining the physiological sensor data 114 x₀ (k) and the motion reference data 126 x₁(k), . . . ,x_{K}(k) as follows:

d(k)=W₀x₀(k)+W₁x₁(k)+ . . . +W_{L}x_{L}(k), with L=N·M

The weights Wᵢ are computed solving a system of linear equations

X^{T}Xw=b,

where X=[x₀, x₁, . . . , x_{L}] is a K×L matrix with xᵢ=[xᵢ(1), xᵢ(2), . . . , xᵢ(K)]^{T}. The vector w contains the weights Wᵢ, and the elements of the prestored normalized correlation vector b, representing the a priory predicted normalized correlations among the vectors xᵢ.

In the shown embodiment, b=[1,0, . . . ,0] for zero correlation is predicted between the artifact-removed physiological sensor data 130 and the motion reference data.

FIG. 4 shows a block diagram of an embodiment of a reconstruction unit 140 for use in the first or second embodiment of the data processing device.

A first reconstruction subunit 410 is configured to receive the artifact-removed physiological sensor data 130 and to use a singular spectrum analysis algorithm as described for the first removal subunit 310. More specifically, the following steps are used to compute the reconstructed components from a mean corrected segment of a motion reference signal, say s'(n) with n ∈{1, . . . N}. Based on the embedded dimension K and the definition L:=N+1−K. Form the L×K trajectory matrix S′=[s′₀, s′₁, . . . , s′_{K}], where s′ₖ are column vectors with elements s′ₖ:=[s′ (k), s′ (k+1) , . . . , s′ (k+L−1)]^{T} Determine the eigenvalues λ′₁≥λ′₂≥. . . λ′_{K}≥0 and eigenvectors v′₁, . . . , v′_{K} of the covariance matrix S′^{T}S′ and project S′ onto the eigenvectors A′:=S′ V′ where A′ is a matrix containing the principal components a′ₖ as columns and V′ a matrix with eigenvectors v′ₖ as columns. The reconstructed components r′ₖ (n) are given by

$${r}_{k}^{′} ( n ) := \frac{1}{{M}_{n}} {\sum }_{m = {L}_{n}}^{{U}_{n}} {a}_{k}^{′} ( n - m + 1 ) {v}_{K}^{′} ( m ) ,$$

with (Mₙ, Lₙ, Uₙ)=(1/n,1, n) for 1≥n≥M−1,

(Mₙ, Lₙ, Uₙ)=(1/M,1, M) for M≥n≥K and

(Mₙ, Lₙ, Uₙ)=(1/(N−n+1) , n−N+M, M) for K+1≥n≥N.

Thereby, the first reconstruction subunit 410 decomposes the artifact-removed physiological data into a plurality of artifact-removed physiological sensor data components r′ₖ (n) 145, which include oscillatory components, varying trends and noise. A sum of these components would result in the artifact-removed physiological data 130. Experiments show that the outlined SSA method works particularly well for the reconstruction subunit 410 with N=512 at a sample rate of 64 Hz and K=128.

Depending on an eigenvalue spectrum of a covariance matrix pertaining to the trajectory matrix, which has column vectors of artifact-removed physiological sensor data samples 130 of a time frame of predetermined length, a component subset of at least two of the artifact-removed physiological sensor data components 145 is chosen in the second reconstruction subunit 420. Thereby, at least two of the artifact-removed physiological sensor data components 145 of highest component amplitudes are selected. The eigenvalues of the covariance matrix serve as a measure for the component amplitude of the artifact-removed physiological sensor data components 145. For the shown embodiments, the two artifact-removed physiological sensor data components 145 with the highest eigenvalues of the respective covariance matrix pertaining to the trajectory matrix are selected by the second reconstruction subunit 420.

In the third reconstruction subunit 430 the two artifact-removed physiological sensor data components 145 of highest component amplitudes are combined by determining the sum of both components. Afterwards, the third reconstruction subunit 430 provides the sum of both components as reconstructed heart beat component data 150.

In an embodiment not shown, the reconstruction unit is further configured to adapt a total number of the artifact-removed physiological sensor data components forming the component subset. The adaptation can depend on the eigenvalue spectra of the covariance matrix determined by the first reconstruction subunit of the reconstruction unit.

In an embodiment not shown, the first reconstruction subunit determines the artifact-removed physiological sensor data components only for the components that belong to those eigenvectors of the covariance matrix which have the at least two largest eigenvalues.

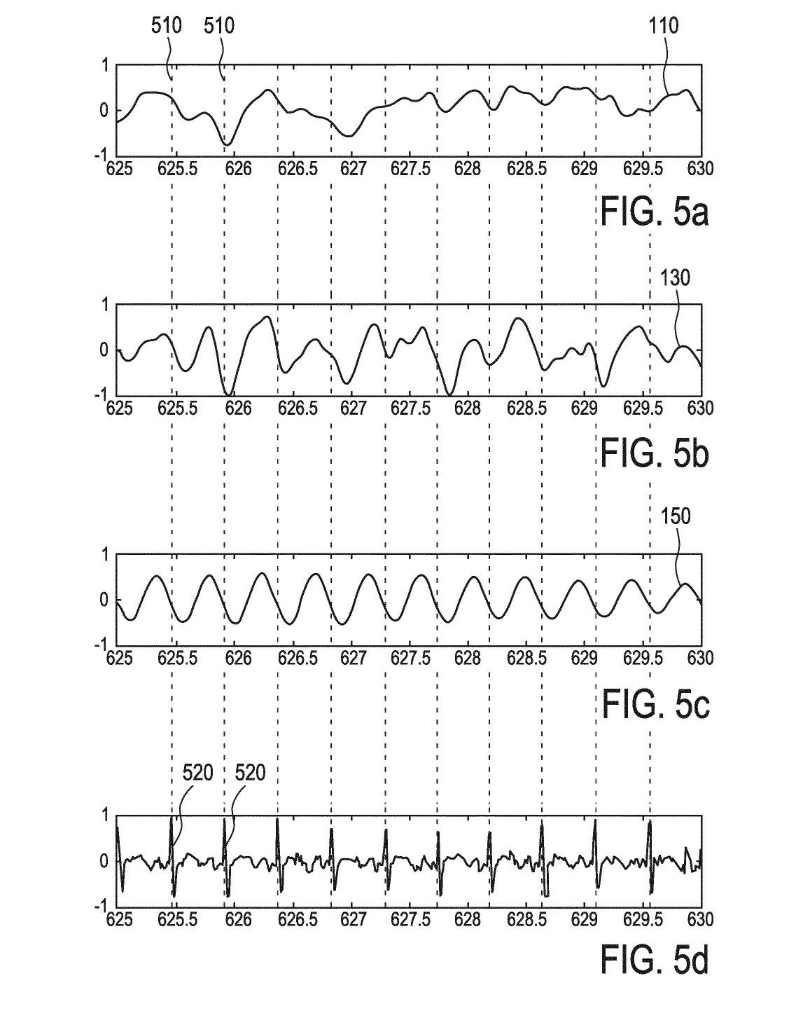

FIGS. 5a-d show sensor data 110 (FIG. 5a), artifact-removed physiological sensor data 130 (FIG. 5b), reconstructed heart beat component data 150 (FIG. 5c) and an electrocardiogram (FIG. 5d) over a time period of 5 sec received at respective units of the data processing device according to the first aspect of the invention.

FIG. 5a shows bandpass filtered PPG data as sensor data 110. The evolution of the amplitude of the sensor data 110 over time shows nearly no periodicity in view of a strong affect of motion artifacts on the sensor data.

FIG. 5b shows that after an artifact removal by the motion-artifact removal unit 120, peaks and dips are more visible, but an interbeat extraction algorithm, based on peaks and dips, would still provide a value of the interbeat interval comprising large errors.

FIG. 5c shows the reconstructed heart beat component data 150 for the sensor data 110 shown in FIG. 5a. It resembles a sinusoidal signal with local peaks and dips removed, which makes an interbeat interval detection of the beat analysis unit 160 much more precise.

FIG. 5d shows amplitudes of an electrocardiogram that corresponds to the sensor data 110 shown in FIG. 5a. The dashed lines 510 provided for the FIGS. 5a to 5d indicate peaks 520 of the electrocardiogram. A comparison of the reconstructed heart beat component data 150 with the peaks 520 of the electrocardiogram shows that the reconstructed heart beat component data corresponds to the peaks 520 of electrocardiogram, while a correspondence between electrocardiogram and artifact-removed physiological sensor data can not be unambiguously observed. This example underlines the advantage of a processing of the reconstruction unit 140 for extracting heart rate information.

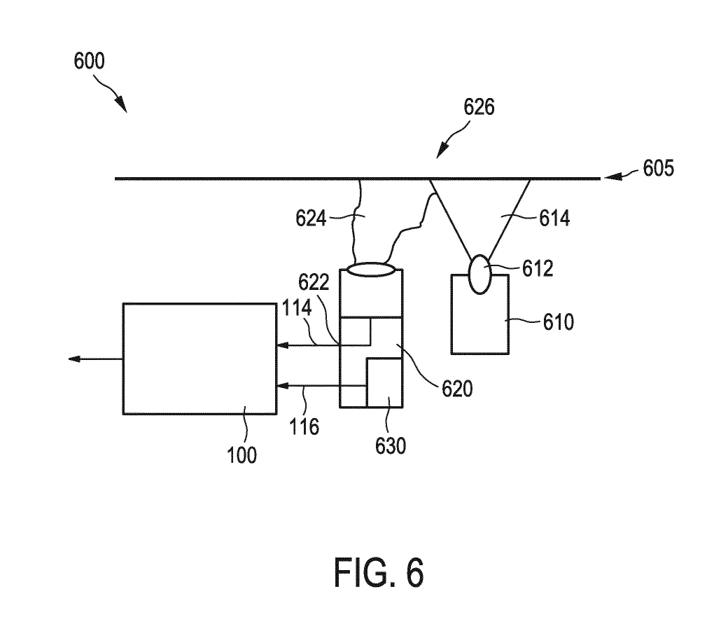

FIG. 6 shows an embodiment of an apparatus 600 for determining heart rate information of a subject of interest 605, according to a second aspect of the invention.

The apparatus 600 comprises an emitter unit 610, a sensor unit 620, an accelerometer 630 and the data processing device 100 according to the first embodiment shown in FIG. 1.

The emitter unit 610 comprises at least one emitter 612, which is configured to emit electromagnetic radiation 614 in at least one spectral channel that allows determining heart rate information. Preferably the spectral channel includes the wavelength region between 540 nm and 560 nm, which provides a particularly high sensitivity to blood volume variations.

The sensor unit 620 is configured to ascertain and provide at its output 622 physiological sensor data 114 that is indicative of an amount of electromagnetic radiation 624 reflected from or transmitted through a sensed region 626 of the subject of interest 605 as a function of time in at least one spectral channel that includes the heart beat component and at least one motion artifact component in a respective spectral region of the electromagnetic spectrum. The sensed region 626 in this embodiment is a tissue of the subject of interest, wherein preferably tissue of the wrist of the subject of interest 605 is used for determining the heart rate information.

The sensor unit 620 further comprises the accelerometer 630, which is arranged and configured to provide motion sensor data 116 in form of accelerometer data. The motion sensor data 116 is indicative of a velocity or an acceleration of the sensed region as a function of time.

The sensor data 110 formed by the physiological sensor data 114 and the motion sensor data 116 is provided to the data processing device 100 according to the first embodiment shown in FIG. 1.

The apparatus of this embodiment is preferably situated in a wrist band, such that the emitter unit can emit the electromagnetic radiation 614 at the wrist of the user of the apparatus. Similar embodiments can be situated in chest belts, home monitoring systems or other medical devices that are used to determine the heart rate information of a user of the device.

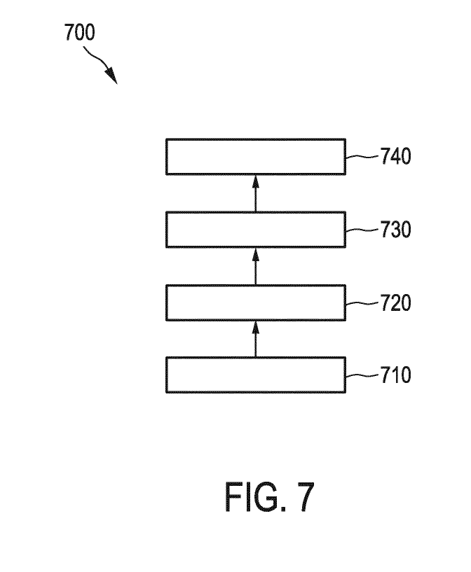

FIG. 7 shows a flow diagram of a first embodiment of a data processing method 700 for extracting heart rate information of a subject of interest from time-dependent sensor data, according to a third aspect of the invention.

The data processing method comprises four steps as described in the following:

In a step 710 time-dependent sensor data is received that includes physiological sensor data comprising a heart beat component and at least one motion artifact component, and that further includes motion sensor data, which is indicative of a velocity or an acceleration of a sensed region of the subject of interest.

A further step 720 comprises a decomposing of the motion sensor data into at least two components of decomposed motion sensor data, a determining of at least two different sets of motion reference data, based on the at least two components of decomposed motion sensor data, a combining of the physiological sensor data and the sets of motion reference data, and a providing of artifact-removed physiological sensor data.

A further step 730 comprises a receiving of the artifact-removed physiological sensor data, a decomposing of the artifact-removed physiological sensor data into a plurality of artifact-removed physiological sensor data components with associated component amplitudes, a combining of a component subset comprising at least two of the artifact-removed physiological sensor data components with highest component amplitudes, and a providing of the combined component subset as reconstructed heart beat component data.

A final step 740 comprises a receiving of the reconstructed heart beat component data, a determining of an interbeat interval from the heart beat component data as the heart rate information, and a providing of a heart rate information signal based on the determined heart rate information.

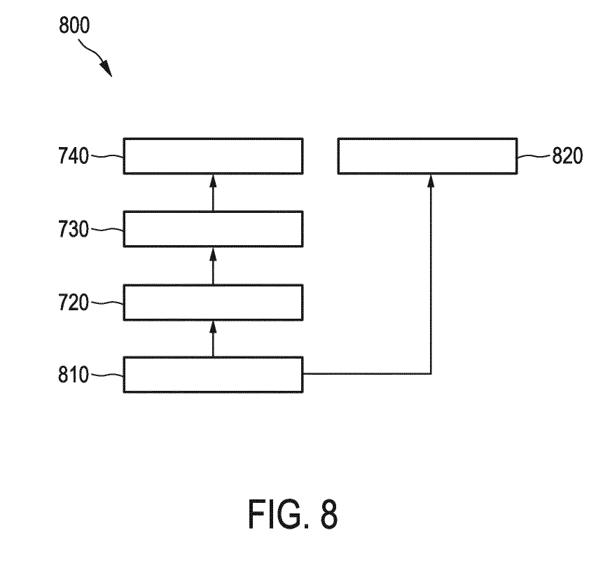

FIG. 8 shows a flow diagram of a second embodiment of the data processing method 800 according to the third aspect of the invention.

Compared to the first embodiment shown in FIG. 7, the second embodiment additionally comprises for the first step 810 an ascertaining of a motion level indicative of a current amount of velocity or acceleration of the sensed region of the subject of interest.

As long as the motion level value does not exceed a predetermined motion level threshold, the next and final step 820 is a determining the interbeat interval from only the sensor data or the physiological sensor data. If the motion level value does exceed a predetermined motion level threshold, the further steps of the method are the steps 720, 730 and 740 of the first embodiment shown in FIG. 7.

In summary, a data processing device is provided for determining heart rate information regarding a subject of interest from time-dependent sensor data that includes physiological sensor data comprising a heart beat component and at least one motion artifact component, and that further includes motion sensor data. A reconstruction unit receives artifact-removed physiological sensor data and decomposes it into a plurality of artifact-removed physiological sensor data components with associated component amplitudes, and provides reconstructed heart beat component data as a combination of a component subset of at least two of the artifact-removed physiological sensor data components of highest component amplitudes. A beat analysis unit receives the reconstructed heart beat component data and determines an interbeat interval from the heart beat component data as the heart rate information, and provides a heart rate information signal based on the determined heart rate information.

While the present invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In particular the invention is not restricted to a particular way for obtaining motion sensor data. The invention is furthermore not restricted to medical applications.

In the claims, the word “comprising” does not exclude other elements or steps, and the indefinite article “a” or “an” does not exclude a plurality. The combination of elements by the word “or” does not exclude an element but clarifies that every combination of the combined elements is possible.

A single step or other units may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A data processing device for determining heart rate information regarding a subject of interest from time-dependent sensor data that includes physiological sensor data comprising a heart beat component and at least one motion artifact component, and that further includes motion sensor data, which is indicative of a velocity or an acceleration of a sensed region of the subject of interest, the data processing device comprising:
a motion-artifact removal unit, configured to receive the sensor data, to apply a motion-artifact-removal algorithm to the physiological sensor data using the motion sensor data and to provide artifact-removed physiological sensor data;
a reconstruction unit, configured to receive the artifact-removed physiological sensor data and to decompose the artifact-removed physiological sensor data into a plurality of artifact-removed physiological sensor data components with associated component amplitudes, and to provide reconstructed heart beat component data as a combination of a component subset of at least two of the artifact-removed physiological sensor data components of highest component amplitudes; and
a beat analysis unit, configured to receive the reconstructed heart beat component data, to determine an interbeat interval from the heart beat component data as the heart rate information, and to provide a heart rate information signal based on the determined heart rate information.

2. The data processing device according to claim 1, wherein the data processing device is further configured to determine from the motion sensor data or to receive a motion level indicator indicative of whether a motion level derived from a current amount of velocity or acceleration of the sensed region of the subject of interest exceeds a predetermined motion level threshold; and wherein the beat analysis unit is further arranged and configured only as long as according to the motion level indicator the motion level does not exceed a predetermined motion level threshold, to receive the physiological sensor data and to determine the interbeat interval from only the physiological sensor data.

3. The data processing device according to claim 1, which is configured to receive the physiological sensor data in form of PPG data, which is indicative of an amount of electromagnetic radiation reflected from or transmitted through the sensed region of the subject of interest as a function of time in at least one first spectral channel that is sensitive to blood volume variations in the sensed region.

4. The data processing device according to claim 3, which is configured to receive the time dependent motion sensor data at least partly in form of optical motion sensor data, which is indicative of an amount of electromagnetic radiation reflected from or transmitted through the sensed region of the subject of interest as a function of time in at least one further spectral channel that is not sensitive to blood volume variations in the sensed region.

5. The data processing device according to claim 1, which is configured to receive the time dependent motion sensor data at least partly in form of acceleration data provided by an accelerometer.

6. The data processing device according to claim 1, which is configured to structure the time-dependent sensor data into frames containing sensor data pertaining to predetermined time spans, wherein
the motion-artifact removal unit is configured to apply the motion-artifact-removal algorithm to the physiological sensor data on a frame-by-frame basis, and wherein
the reconstruction unit is configured to decompose the artifact-removed physiological sensor data and to combine the component subset of the at least two of the artifact-removed physiological sensor data components on a frame-by-frame basis.

7. The data processing device of claim 1, wherein the reconstruction unit is configured to adapt a total number of the artifact-removed physiological sensor data components forming the modeling component subset in accordance with at least one predetermined adaptation control criterion.

8. The data processing device according to claim 1, wherein the motion-artifact removal unit is configured to decompose the motion sensor data into at least two components of decomposed motion sensor data, to determine, based on the at least two components of decomposed motion sensor data, at least two different sets of motion reference data, and to combine the physiological sensor data and the sets of motion reference data so as to provide the artifact-removed physiological sensor data.

9. The data processing device according to claim 8, wherein the motion-artifact removal unit is configured to decompose the motion sensor data into the at least two components of decomposed motion sensor data using a singular spectrum analysis algorithm.

10. The data processing device according to claim 1, wherein the reconstruction unit is configured to decompose the artifact-removed physiological sensor data into the plurality of artifact-removed physiological sensor data components using a singular spectrum analysis algorithm.

11. The data processing device according to claim 10, wherein the reconstruction unit is configured to determine eigenvalues and eigenvectors of a covariance matrix pertaining to a trajectory matrix, which has column vectors of artifact-removed physiological sensor data samples of a time frame of predetermined length, and to project the trajectory matrix onto the eigenvectors so as to determine the artifact-removed physiological sensor data components and their associated component amplitudes.

12. An apparatus for determining heart rate information regarding a subject of interest, the apparatus comprising:
an emitter unit, which comprises at least one emitter, which is configured to emit electromagnetic radiation at a sensed region of the subject of interest in at least one spectral channel that allows ascertaining physiological sensor data comprising a heart beat component,
a sensor unit, which is configured to ascertain and provide at its output physiological sensor data that is indicative of an amount of electromagnetic radiation reflected from or transmitted through a sensed region of the subject of interest as a function of time in at least one spectral channel and that includes the heart beat component and at least one motion artifact component, and to ascertain motion sensor data that is indicative of a velocity or an acceleration of the sensed region as a function of time; and
a data processing device according to claim 1.

13. The apparatus according to claim 12, wherein the sensor unit comprises an accelerometer, which is arranged and configured to provide the motion sensor data in form of accelerometer data.

14. A data processing method for determining heart rate information regarding a subject of interest from time-dependent sensor data, the data processing method comprising:
receiving time-dependent sensor data that includes physiological sensor data comprising a heart beat component and at least one motion artifact component, and that further includes motion sensor data, which is indicative of a velocity or an acceleration of a sensed region of the subject of interest;
applying a motion-artifact-removal algorithm to the physiological sensor data using the motion sensor data and providing artifact-removed physiological sensor data;
receiving the artifact-removed physiological sensor data, decomposing the artifact-removed physiological sensor data into a plurality of artifact-removed physiological sensor data components with associated component amplitudes, combining a component subset comprising at least two of the artifact-removed physiological sensor data components with highest component amplitudes, and providing the combined component subset as reconstructed heart beat component data; and
receiving the reconstructed heart beat component data, determining an interbeat interval from the heart beat component data as the heart rate information, and providing a heart rate information signal based on the determined heart rate information.

15. A computer program comprising program code means for causing a computer to perform the method as claimed in claim 14 when said computer program is executed on the computer.
